(19) United States
(12) Patent Application Publication
(10) Pub. No.: US 2004/0092532 A1
(43) Pub. Date: May 13, 2004

(54) Use of p38 inhibitors for the treatment of smoke inhalation

(76) Inventors: Don E Griswold, North Wales, PA (US); David C Underwood, King of Prussia, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/399,580

(22) PCT Filed: Oct. 19, 2001
(86) PCT No.: PCT/US01/50429

(51) Int. Cl. A61K 31/506; A61K 31/405; A61K 31/56; A61K 31/485; A61K 31/415

(52) U.S. Cl. 514/256; 514/282; 514/341; 514/420; 514/406; 514/171; 514/161; 514/570

(57) ABSTRACT

The present invention is directed to the novel use of a CSBP/p38 inhibitor for the treatment, including prophylaxis of smoke induced pathology resulting from acute and chronic inflammation in the lung.

### FIELD OF INVENTION

The present invention relates to the use of a CSBP/p38 inhibitor in the treatment of inflammation enhanced cough related disorders that are CSBP/p38 mediated.

### BACKGROUND OF THE INVENTION

Lung or lung airway inflammatory response is thought to be orchestrated by macrophage- and epithelial-derived cytokines, such as TNF-α and IL-1β which enhance the expression of vascular adhesion molecules (ICAM-1, E-selectin) and neutrophil chemotaxins or chemokines, such as IL-8, to generate the release of destructive oxidants and proteases [Warner et al., Am J. Respir Crit Care Med. 160:S1-S79 (1999)].

It is well known that inflammatory cytokines (TNF-α,IFN-γ,IL-4,IL-5) and chemokines (IL-8, RANTES, eotaxin) are capable of regulating or supporting chronic airway inflammation [Barnes et al., Pharmacol Rev. 50:515-596 (1998)]. The production and action of many of the potential mediators of airway inflammation have been shown to be dependent upon the stress induced MAP kinase or p38 kinase (p38 MAPK) cascade [Foltz et al., J. Biol. Chem. 27:3296-3301 (1997)]. A variety of inflammatory mediators activate p38 MAPK which may then activate downstream targets of the MAPK system including other kinases or transcription factors, thus creating the potential for an amplified inflammatory process in the lung.

By interfering with the biochemical processes produced in this cascade, there represents a viable and new use for intervention with an inhibitor of CSBP/p38. This invention is directed to the novel discovery of treatment, including prophylaxis, of hypertussive activity in mammals afflicted with increased eosinophilic, or inflammation in the airways and with cough.

### BRIEF DESCRIPTION OF THE DRAWINGS

FIG. 1 demonstrates a Citric Acid Induced Cough Model.

FIG. 2 demonstrates an Antigen- or LTD4-Induced Hypertussive Model in the Guinea Pig

FIG. 3 demonstrates Effects of Dextromethorphan or Codeine On Citric Acid-Induced Cough in Guinea Pigs.

FIG. 4 demonstrates the effects of Compound I, trans-1-(4-Hydroxycyclohexyl)-4-(4fluorophenyl)-5-[(2-methoxy)pyrimidin-4-yl]imidazole on Citric Acid induced Cough

FIG. 5 demonstrates the effects of Compound I on Antigen—Induced Hypertussive Activity to Citric Acid in Guinea Pigs

### SUMMARY OF THE INVENTION

The present invention relates to the use of a CSBP/p38 kinase inhibitor for the treatment, including prophylaxis, of the hypertussive activity associated with resulting airway inflammation and/or cough in a mammal in need thereof.

The present invention also relates to use of a CSBP/p38 kinase inhibitor for the treatment, including prophylaxis, of the inflammation enhanced cough related disorders in a mammal in need thereof.

The present invention is also directed to the use of a CSBP/p38 kinase inhibitor in eosinophilic bronchitis, and in cough variant asthma.

### DETAILED DESCRIPTION OF THE INVENTION

IL-1, TNF, and other cytokines affect a wide variety of cells and tissues and these cytokines, as well as other leukocyte derived cytokines, are important and critical inflammatory mediators of a wide variety of disease states and conditions. Thus inhibition of these cytokines is of benefit in controlling, reducing and alleviating many of these disease states.

In particular, the present invention is directed to the treatment, including prophylaxis, of eosinophilic inflammation in the airways and cough. The invention is also directed to treatment, including prophylaxis where appropriate for eosinophilic bronchitis (as this differs from asthma) and for the treatment, including prophylaxis of cough variant asthma. These disorders may be directed to treatment of the airway induced inflammation which is secondary to other respiratory disorders such as viral infections that exacerbate asthma (induced by such infections), chronic bronchitis, chronic obstructive pulmonary disease, otitis media, and sinusitis. A respiratory viral infection treated in conjunction with the smoke related airway inflammation may also be associated with a secondary bacterial infection, such as otitis media, sinusitis, or pneumonia.

The hypertussive or inflammation enhanced cough related disorders may either be a direct result of or an association with eosinophilia activity. It may also be a result of, or associated with the blocking production of certain cytokines which may mediate these phenomena.

For use herein treatment may include prophylaxis for use in a treatment group who may be susceptible to such airway inflammation, and/or cough. It may also include reducing the symptoms of, ameliorating the symptoms of, reducing the severity of, reducing the incidence of, or any other change in the condition of the patient, which improves the therapeutic outcome.

The mechanism of action for inhibition of a cytokine by a cytokine suppressive anti-inflammatory drug (CSAID) is well known in the art. The present invention will demonstrate that CSAID inhibitors are useful in the treatment of eosinophilic inflammation of the airways and cough.

Clinically, eosinophilic bronchitis presents as chronic cough and sputum eosinophilia, but without the abnormalities of airway function seen in asthma. In contrast to cough in patients without sputum eosinophilia, the cough responds to anti-inflammatory therapy, such as inhaled corticosteroids (Niimi et al., Eosinophilic inflammation in cough variant asthma, European Respiratory Journal. 11(5):1064-9, (1998)).

Patients with cough-variant asthma may also have the following criteria: (1) have not been previously diagnosed as having asthma; (2) complain of a cough of at least a 3-week duration; (3) do not complain of wheezing, shortness of breath, or chest tightness; (4) have normal results of physical examinations; (5) have normal or nearly normal results of spirometry; (6) have evidence of bronchial hyper-responsiveness during bronchoprovocation challenge testing; and (7) have a favorable response to asthma medications (Irwin et al., Interpretation of positive results of a methacholine inhalation challenge and 1 week of inhaled bronchodilator use in diagnosing and treating cough-variant asthma (Archives of Internal Medicine. 157(17):1981-1987, (1997)).

Unlike conventional antitussive agents, such as codeine or dextromethorphan, a p38 kinase inhibitor appears to have no direct antitussive activity, but reduces the airway eosinophilia and normalizes the hypertussive state. Therefore, use of a p38 inhibitor will reduce the added coughs, or hypertussive state, back to a normal level, which can be suitably treated with conventional agents and/or therapies as appropriate. Use of the p38 inhibitors will allow for the maintenance of patients who are subject to increased cough responsiveness, especially unproductive cough, due to other underlying disorders or treatments. This increased cough responsiveness may be modulated, or decreased by use of this innovative anti-inflammatory therapy.

Suitable CSAID compounds are well known in the art, and an assay for determining CBSP/p38 inhibition is also readily available using assays disclosed in the below noted patents or applications. For instance, see U.S. Pat. Nos. 5,716,972, 5,686,455, 5,656,644, 5,593,992, 5,593,991, 5,663,334, 5,670,527, 5,559,137, 5,658,903, 5,739,143, 5,756,499, and 5,716,955; WIPO publications WO 98/25619, WO 97/25048, WO 99/01452, WO 97/25047, WO 99/01131, WO 99/01130, WO 97/33883, WO 97/35856, WO 97/35855, WO 98/06715, WO 98/07425, WO 98/28292, WO 98/56377, WO 98/07966, WO 99/01136, WO 99/17776, WO 99/01131, WO 99/01130, WO 99/32121, WO 00/26209, WO 99/58502, WO 99/58523, WO 99/57101, WO 99/61426, WO 99/59960, WO 99/59959, WO 00/18738, WO 00/17175, WO 99/17204, WO 00/20402, WO 99/64400, WO 00/01688, WO 00/07980, WO 00107991, WO 00/06563, WO 00/12074, WO 00/12497, WO 00/31072, WO 00/31063, WO 00/23072, WO 00/31065, WO 00/35911, WO 00/39116, WO 00/43384, WO 00/41698, WO 97/36587, WO 97/47618, WO 97/16442, WO 97/16441, WO 97/12876, WO 98/7966, WO 98/56377, WO 98/22109, WO 98/24782, WO 98/24780, WO 98/22457, WO 98/52558, WO 98/52941, WO 98/52937, WO 98/52940, WO 98/56788, WO 98/27098, WO 99/00357, WO 98/47892, WO 98/47899, WO 99/03837, WO 99/01441, WO 99/01449, WO 99/03484, WO 95/09853, WO 99/15164, WO 98/50356, WO 95/09851, WO 95/09847, WO 95/09852, WO 92/12154, WO 94/19350, DE 19842833, JP 2000 86657, and De Laszlo et al., Bioorg. Med. Chem. Lett 8 (1998) 2689-2694 whose disclosures are all incorporated herein by reference in their entirety.

Preferred compounds of this invention include those contained in WO 99/01131, and a representative genus is described below. Also preferred for use herein are the compounds disclosed in WO 99/61426 Scios, Inc.; and those compounds disclosed in WO 98/27098 containing the compound known as VX-745; (also known as 5-(2,6-Dichloro-phenyl)-2-(2,4-difluoro-phenylsulfanyl)-1,7,8a-triaza-naphthalen-6-one), the Johnson & Johnson compound RWJ-68354 disclosed in WO 98/47899, RPR compound RPR-200765A, the Zeneca compound ZM 336372 disclosed in WO 99/15164; the Sugen compound SU 4984 disclosed in WO 98/50356. A review of various inhibitors of p38 kinase is taught in Boehm et al., Exp. Opin. Ther. Patents 10(1):25-37 (2000).

Compounds of Formula (I) are represented by the formula:

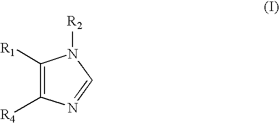

wherein

R₁ is 4-pyridyl, pyrimidinyl, 4-pyridazinyl, 1,2,4-triazin-5-yl, quinolyl, isoquinolinyl, or quinazolin-4-yl ring, which ring is substituted with Y-Rₐ and optionally with an additional independent substituent selected from C₁₋₄ alkyl, halogen, hydroxyl, C₁₋₄ alkoxy, C₁₋₄ alkylthio, C₁₋₄ alkylsulfinyl, CH₂OR₁₂, amino, mono and di-C₁₋₆ alkyl substituted amino, an N-heterocyclyl ring which ring has from 5 to 7 members and optionally contains an additional heteroatom selected from oxygen, sulfur or NR₁₅, N(R₁₀)C(O)R_{b} or NHRₐ;

Y is oxygen or sulfur;

R₄ is phenyl, naphth-1-yl or naphth-2-yl, or a heteroaryl, which is optionally substituted by one or two substituents, each of which is independently selected, and which, for a 4-phenyl, 4-naphth-1-yl, 5-naphth-2-yl or 6-naphth-2-yl substituent, is halogen, cyano, nitro, C(Z)NR₇R₁₇, C(Z)OR₁₆, (CR₁₀R₂₀)ᵥCOR₁₂, SR₅, SOR₅, OR₁₂, halo-substituted-C₁₋₄ alkyl, C₁₋₄ alkyl, ZC(Z)R₁₂, NR₁₀C(Z)R₁₆, or (CR₁₀R₂₀)ᵥNR₁₀R₂₀ and which, for other positions of substitution, is halogen, cyano, C(Z)NR₁₃R₁₄, C(Z)OR₃, (CR₁₀R₂₀)_{m″}COR₃, S(O)ₘR₃, OR₃, halo-substituted C₁₋₄ alkyl, C₁₋₄ alkyl, (CR₁₀R₂₀)_{m″}NR₁₀C(Z)R₃, NR₁₀S(O)_{m′}R₈, NR₁₀S(O)_{m′}NR₇R₁₇, ZC(Z)R₃ or (CR₁₀R₂₀)_{m″}NR₁₃R₁₄;

Z is oxygen or sulfur;

n is an integer having a value of 1 to 10;

m is 0, or the integer 1 or 2;

m′ is an integer having a value of 1 or 2,

m″ is 0, or an integer having a value of 1 to 5;

v is 0, or an integer having a value of 1 or 2;

R₂ is —C(H) (A) (R₂₂);

A is an optionally substituted aryl, heterocyclyl, or heteroaryl ring, or A is a substituted C₁₋₁₀ alkyl;

R₂₂ is an optionally substituted C₁₋₁₀ alkyl;

Rₐ is aryl, arylC₁₋₆alkyl, heterocyclic, heterocyclylC₁₋₆ alkyl, heteroaryl, heteroarylC₁₋₆alkyl, wherein each of these moieties may be optionally substituted;

R_{b} is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl, wherein each of these moieties may be optionally substituted;

R₃ is heterocyclyl, heterocyclylC₁₋₁₀ alkyl or R₈;

R₅ is hydrogen, C₁₋₄ alkyl, C₂₋₄ alkenyl, C₂₋₄ alkynyl or NR₇R₁₇, excluding the moieties SR₅ being SNR₇R₁₇ and SOR₅ being SOH;

R₆ is hydrogen, a pharmaceutically acceptable cation, C₁₋₁₀ alkyl, C₃₋₇ cycloalkyl, aryl, arylC₁₋₄ alkyl, heteroaryl, heteroarylC₁₋₄ alkyl, heterocyclyl, aroyl, or C₁₋₁₀ alkanoyl;

R₇ and R₁₇ is each independently selected from hydrogen or C₁₋₄ alkyl or R₇ and R₁₇ together with the nitrogen to which they are attached form a heterocyclic ring of 5 to 7 members which ring optionally contains an additional heteroatom selected from oxygen, sulfur or NR₁₅;

R₈ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylC₁₋₁₀ alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈, (CR₁₀R₂₀)ₙNR₁₃R₁₄; wherein the aryl, arylalkyl, heteroaryl, heteroaryl alkyl may be optionally substituted;

R₉ is hydrogen, C(Z)R₁₁ or optionally substituted C₁₋₁₀ alkyl, S(O)₂R₁₈, optionally substituted aryl or optionally substituted aryl-C₁₋₄ alkyl;

R₁₀ and R₂₀ is each independently selected from hydrogen or C₁₋₄ alkyl;

R₁₁ is hydrogen, C₁₋₁₀ alkyl, C₃₋₇ cycloalkyl, heterocyclyl, heterocyclyl C₁₋₁₀alkyl, aryl, arylC₁₋₁₀ alkyl, heteroaryl or heteroarylC₁₋₁₀ alkyl, wherein these moieties may be optionally substituted;

R₁₂ is hydrogen or R₁₆;

R₁₃ and R₁₄ is each independently selected from hydrogen or optionally substituted C₁₋₄ alkyl, optionally substituted aryl or optionally substituted aryl-C₁₋₄ alkyl, or together with the nitrogen which they are attached form a heterocyclic ring of 5 to 7 members which ring optionally contains an additional heteroatom selected from oxygen, sulfur or NR₉;

R₁₅ is R₁₀ or C(Z)—C₁₋₄ alkyl;

R₁₆ is C₁₋₄ alkyl, halo-substituted-C₁₋₄ alkyl, or C₃₋₇ cycloalkyl;

R₁₈ is C₁₋₁₀ alkyl, C₃₋₇ cycloalkyl, heterocyclyl, aryl, aryl₁₋₁₀alkyl, heterocyclyl, heterocyclyl-C₁₋₁₀alkyl, heteroaryl or heteroaryl₁₋₁₀alkyl;

or a pharmaceutically acceptable salt thereof.

R₂ is a substituted alkyl derivative. It is recognized that the first methylene carbon in this chain is a tertiary carbon, and it will contain one hydrogen moiety. This methylene group will have has two additional substituents, an R₂₂ moiety and an A moiety, —C(H)(A)(R₂₂). Both A and R₂₂ may not be unsubstituted C₁₋₁₀ alkyl moieties.

In a preferred embodiment, R₂ is a —C(AA₁)(A) moiety, wherein AA₁ is the R₂₂ moiety, but is specifically the side chain residue (R) of an amino acid, as is further described herein.

Suitably, A is an optionally substituted C₃₋₇cycloalkyl, aryl, heteroaryl, or heterocyclic ring, or A is a substituted C₁₋₁₀ alkyl moiety.

When A is an aryl, heteroaryl and heterocyclic ring, the ring may be substituted independently one or more times, preferably, 1 to 3 times by C₁₋₁₀ alkyl; halogen; halo substituted C₁₋₁₀ alkyl, such as CF₃; (CR₁₀R₂₀)ₜOR₁₁; (CR₁₀R₂₀)ₜNR₁₃R₁₄, especially amino or mono- or di-C₁₋₄ alkylamino; (CR₁₀R₂₀)ₜS(O)ₘR₁₈, wherein m is 0, 1 or 2; SH; NR₁₀C(Z)R₃ (such NHCO(C₁₋₁₀ alkyl)); or NR₁₀S(O)ₘR₈ (such as NHSO₂(C₁₋₁₀ alkyl)).

Suitably, t is 0, or an integer of 1 to 4.

When A is an optionally substituted cycloalkyl it is as defined below with the R₂₂ substitution.

When A is an optionally substituted heterocyclyl ring, the ring is preferably a morpholino, pyrrolidinyl, piperazinyl or a piperidinyl ring.

When A is an optionally substituted aryl moiety, it is preferably a phenyl ring.

When A is an optionally substituted heteroaryl ring, it is as defined below in the definition section.

When A is a substituted C₁₋₁₀ alkyl moiety, the alkyl chain may be straight or branched. The chain is substituted independently 1 or more times, preferably 1 to 3 times by halogen, such as fluorine, chlorine, bromine or iodine; halosubstituted C₁₋₁₀ alkyl, such as CF₃; C₃₋₇cycloalkyl, C₁₋₁₀ alkoxy, such as methoxy or ethoxy; hydroxy substituted C₁₋₁₀ alkoxy; halosubstituted C₁₋₁₀ alkoxy, such as OCF₂CF₂H; OR₁₁; S(O)mR₁₈ (wherein m is 0, 1 or 2); NR₁₃R₁₄; C(Z)NR₁₃R₁₄; S(O)m′NR₁₃R₁₄; NR₂₃C(Z)R₁₁; NHS(O)₂R₁₈; C(Z)R₁₁; OC(Z)R₁₁; C(Z)OR₁₁; C(Z)NR₁₁OR₉; N(OR₆)C(Z)NR₁₃R₁₄; N(OR₆)C(Z)R₁₁; C(═NOR₆)R₁₁; NR₂₃C(═NR₁₉)NR₁₃R₁₄; OC(Z)NR₁₃R₁₄; NR₂₃C(Z)NR₁₃R₁₄; or NR₂₃C(Z)OR₁₀.

Preferably A is a C₃₋₇ cycloalkyl, or a C₁₋₆ alkyl, more preferably a C₁₋₂ alkyl, i.e. a methylene or ethylene moiety, more preferably a methylene moiety which is substituted by one of the above noted groups.

Preferably, when A is a C₁₋₁₀ alkyl, it is substituted by OR₁₁ where R₁₁is preferably hydrogen, aryl or arylalkyl; NR₁₃R₁₄; OC(Z)R₁₁; or C(Z)OR₁₁.

More preferably, A is substituted by OR₁₁where R₁₁ is hydrogen.

Suitably, R₂₂ is a C₁₋₁₀ alkyl chain, which chain may be straight or branched and which may be optionally substituted independently, one or more times, preferably 1 to 3 times, by halogen, such as fluorine, chlorine, bromine or iodine; halo substituted C₁₋₁₀ alkyl; C₁₋₁₀ alkoxy, such as methoxy or ethoxy; hydroxy substituted C₁₋₁₀ alkoxy; halosubstituted C₁₋₁₀ alkoxy, such as OCF₂CF₂H; OR₁₁; S(O)ₘR₁₈; NR₁₃R₁₄; C(Z)NR₁₃R₁₄; S(O)m′ NR₁₃R₁₄; NR₂₃C(Z)R₁₁; NHS(O)₂R₁₈; C(Z)R₁₁; OC(Z)R₁₁; C(Z)OR₁₁; C(Z)NR₁₁ OR₉; N(OR₆)C(Z)NR₁₃R₁₄; N(OR₆)C(Z)R₁₁; C(═NOR₆)R₁₁; NR₂₃C(═NR₁₉)NR₁₃R₁₄; OC(Z)NR₁₃R₁₄; NR₂₃C(Z)NR₁₃R₁₄; NR₂₃C(Z)OR₁₀; optionally substituted C₃₋₇ cycloalkyl; optionally substituted aryl, such as phenyl; optionally substituted heteroaryl; or an optionally substituted heterocyclic. The optional substituents on these cycloalkyl, aryl, heteroaryl, and heterocyclic moieties are as defined herein below.

It is noted that those R₂₂ substituent groups which contain carbon as the first connecting group, i.e. C(Z)OR₁₁; C(Z)NR₁₁OR₉, C(Z)R₁₁, C(Z)NR₁₃R₁₄, and C(═NOR₆)R₁₁, may be the sole carbon in alkyl chain. Therefore, the R₂₂ group may, for instance, be a carboxy, an aldehyde, or an amide, as well as being a substituent off a methylene unit, such as carbamoylmethyl, or acetamidomethyl. Preferably R₂₂ is a C₁₋₆ unsubstituted or substituted alkyl group, such as a C₁₋₃ alkylene, such as methyl, ethyl or isopropyl, or a methylene or ethylene moiety substituted by one of the above noted moieties, or as noted above those substituent groups which contain a carbon may substitutent for the first methylene unit of the alkyl chain, such as carboxy, C(O)OR₁₁,C(O)NR₁₃R₁₄, or R₂₂ is an optionally substituted aryl group, such as a benzyl or phenethyl. In other words, R₂₂ can be an optionally substituted alkyl group, or R₂₂ can be C(Z)OR₁₁, C(Z)NR₁₁OR₉, C(Z)R₁₁, C(Z)NR₁₃R₁₄, or C(═NOR₆)R₁₁

Preferably R₂₂ is a C₁₋₆ unsubstituted or substituted alkyl group, more preferably a C₁₋₂ alkylene chain, such as a methylene or ethylene moiety, more preferably methylene.

Preferably the alkyl chain is substituted by OR₁₁, where R₁₁ is preferably hydrogen, aryl or arylalkyl; S(O)mR₁₈, where m is 0 and R₁₈ is a C₁₋₆ alkyl; or an optionally substituted aryl, i.e. a benzyl or phenethyl moiety.

More preferably, R₂₂ is phenyl, benzyl, CH₂OH, or CH₂—O-aryl.

Preferably, one or both of A and R₂₂ contain hydroxy moieties, such as in C₁₋₆ alkyl OR₁₁, wherein R₁₁ is hydrogen, i.e.CH₂CH₂OH.

Suitably, when AA₁ is the (R) side chain residue of an amino acid, it is a C₁₋₆ alkyl group, which may be straight or branched. This means the R group off the core amino acid of the structure R—C(H)(COOH)(NH₂). The R residue term is for example, CH₃ for alanine, (CH₃)₂CH— for valine, (CH₃)₂CH—CH₂— for leucine, phenyl-CH₂— for phenylalanine, CH₃—S—CH₂—CH₂— for methionine, etc. All generally recognized primary amino acids are included in this groups, such as but not limited to, alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine, hydroxylysine, methylhistidine, and other naturally occurring amino acids not found in proteins, such as β-alanine, γ-aminobutyric acid, homocysteine, homoserine, citrulline, ornithine, canavanine, djenkolic acid, and β-cyanoalanine, or other naturally occurring non-mammalian amino acids.

Preferably AA₁ is the residue of phenylalanine, or alanine. Preferably, A is a hydroxy substituted C₁₋₁₀ alkyl, and R₂₂ is a C₁₋₁₀ alkyl or a hydroxy substituted C ₁₋₁₀ alkyl.

For further definitions please refer to the descriptions in WO 99/01131, or in WO 99/01136, supra.

A preferred compound for use herein is 1-(1,3-Dihydroxyprop-2-yl)-4-(4fluorophenyl)-5-(2-phenoxypyrimidin-4-yl)imidazole, or a pharmaceutically acceptable salt thereof.

Other suitable compounds for use herein include but are not limited to, trans-1(4-Hydroxycyclohexyl)-4-(4-fluorophen [(2-methoxy)pyrimidin-4-yl]imidazole; 1-(4-Piperidinyl)-4-(4-fluorophenyl)-5-(2-methoxy-4-pyridinyl)imidazole; or (4-Fluorophenyl)-2-(4-methylsulfinylphenyl)-5-(4-pyridyl)-imidazole.

Methods of using and dosage amounts are the same as those disclosed in the references cited above. See for instance, Adams et al., U.S. Pat. No. 5,756,499, issued 26 May 1998. In order to use a compound of formula (I) or a pharmaceutically acceptable salt thereof in therapy, it will normally be formulated into a pharmaceutical composition in accordance with standard pharmaceutical practice.

For all methods of use disclosed herein (or the compounds of Formula (I) and other CSAID compounds), suitably, the daily oral dosage regimen will be from about 0.1 to about 80 mg/kg of total body weight, preferably from about 0.2 to 30 mg/kg, more preferably from about 0.5 mg to l5mg. The daily parenteral dosage regimen about 0.1 to about 80 mg/kg of total body weight, preferably from about 0.2 to about 30 mg/kg, and more preferably from about 0.5 mg to 15mg/kg. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day.

The novel use of CSAID compounds herein may also be used in association with the veterinary treatment of mammals, other than humans, in need of such inhibition of CSBP/p38 or cytokine inhibition.

The CSBP/p38 inhibitor may also be administered with a second therapeutic agent, such as a generally accepted anti-tussive agents, such as codeine and dextromethorphan; a PDE4 inhibitor, such as cilomilast; non-sedating antihistamines, such as loratadine (Claritin®), descarboethoxyloratadine (DCL), fexofenadine (Allegra®), and cetirizine hydrochloride (Zyrtec®) etc.; a steroid, such as dexamethasone, prednisone, or prenisolone, etc.; various antibiotics, such as the quinolones, cephalosporins, P-lactamase inhibitors, etc.; anti-inflammatory agents, such as an NSAID, a COX-1 or COX-2 inhibitor, ASA, or indomethacin, etc.

It is recognized that the above noted agents may be administered as immediate release, or as extended release dosage forms, either together with a suitable CSAID compound, or separately. The compositions may be administered sequentially, in combination with, or contemporaneously with a CSAID agent. The administration route of the second agent may also differ from that of the CSAID agent, and hence the dosing schedule may vary accordingly.

Cetirizine HCl manufacture and dosing is described in U.S. Pat. No 4,525,358; fexofenadine manufacture and dosing is described in U.S. Pat. Nos. 4,524,129; 5,375,693; 5,578,610; 5,855,912; 5,932,247; and 6,037,353. Loratadine and DCL manufacture and dosing are described in U.S. Pat. Nos. 4,282,233; 4,371,516; 4,659,716; 4,863,931; 5,314,697; and 5,595,997.

Zamanivar dosing is disclosed in U.S. Pat. Nos. 4,627,432; 4,778,054; 4,811,731; 5,035,237; 5,360,817; and 5,648,379. Oseltamivir dosing is disclosed in U.S. Pat. Nos. 5,763,483; 5,866,601; and 5,952,375.

The CSPB/p38 inhibitor may be administered systemically or non-systemically, such as orally, bucally, topically (intranasal) or via inhalation (aerosol), or both topically and via inhalation.

As noted above, a second therapeutic agent may also be administered by any suitable means, including parenteral, suppository, etc. which means of administration is not necessarily by the same route, nor concurrent therewith.

As used herein “topically” shall include non-systemic administration. This includes the application of a compound externally to the epidermis or the buccal cavity and/or the instillation of such a compound into the ear, eye and nose.

As used herein “systemic administration” refers to oral, intravenous, intraperitoneal and intramuscular administration, subcutaneous intranasal, intrarectal, or intravaginal.

It will be recognized by one of skill in the art that the optimal quantity and spacing of individual dosages of a CSBP/p38 inhibitor will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a CSBP/p38 inhibitor given per day for a defined number of days, can be ascertained by those skilled in the art using conventional course of treatment determination tests.

### BIOLOGICAL EXAMPLES

Described below is an example of how to determine the usefulness of p38 inhibitors in the treatment of hypertussive disorders or inflammation enhanced cough.

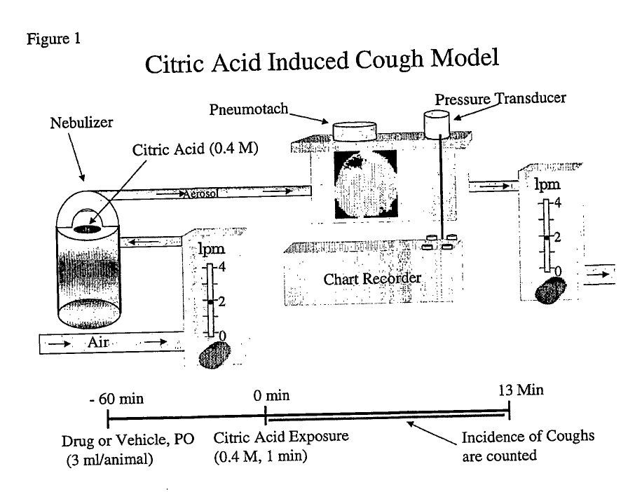

The directed antitussive activity of the compound in question if first assessed, by a 10 to 30 minute pretreatment period by intraperitoneal injection or a 1 hour pretreatment period for oral administration. The animals (guinea pigs) are then subjected to an inhaled citric acid-induced cough challenge. The Citric Acid Induced Cough Model is shown in FIG. 1.

The tool compound chosen for a p38 MAP kinase inhibitor in this inflammatory response, was trans-1-(4-Hydroxycyclohexyl)-4-(4-fluorophenyl)-5-[(2methoxy)pyrimidin-4-yl]imidazole), also referred to as Compound I herein.

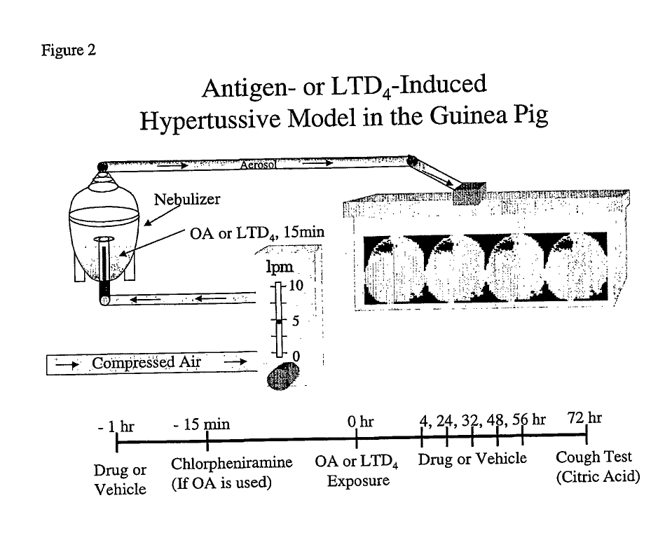

The effects of the compound are then assessed on the hypertussive response that occurs 72 hours post aerosol exposure to antigen or LTD4 exposure. Treatment of the animals occurs with the drug prior and/or after antigen or LTD4 challenge, but not on the day of citric acid challenge. The antigen or LTD4 induced hypertussive model is shown in FIG. 2.

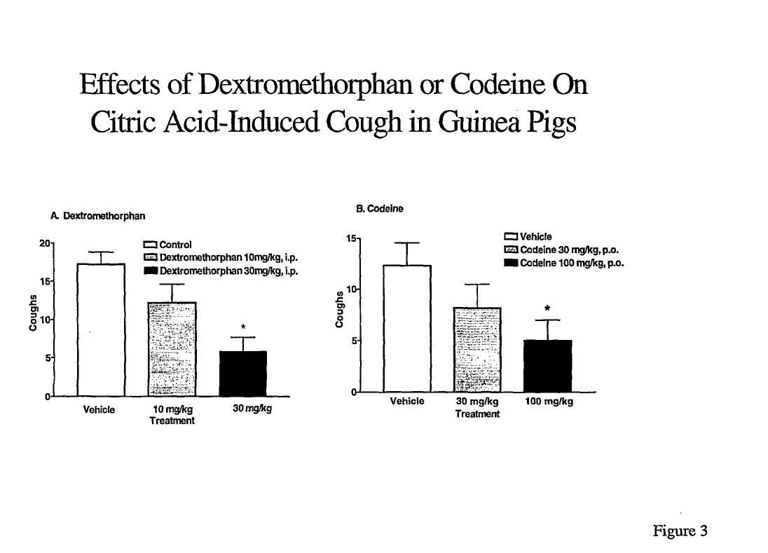

The effects of known antitussive agents, dextromethorphan and codeine on Citric Acid Induced Cough in Guinea Pigs is shown in FIG. 3.

Inhalation of citric acid (CA; 0.4% for 1 minute) induced 11 to 15 coughs, during the exposure and 12-minute monitoring period in conscious guinea pigs. Exposure of sensitized animals to inhaled ovalbumin resulted in a hypertussive state (50-80% increase in CA-induced cough incidence) for several days, which positively correlated with airway esoinophilia determined by bronchoalveolar lavage.

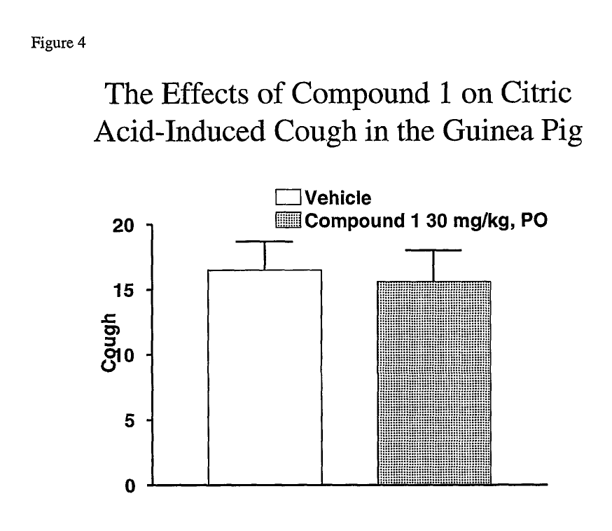

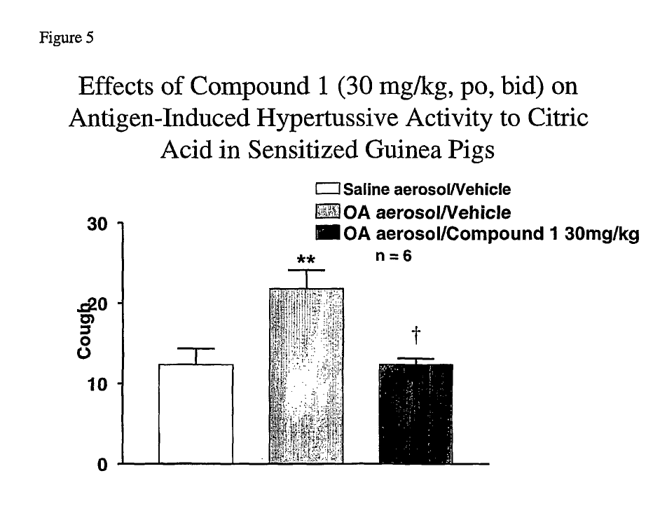

Compound I, has no direct antitussive effect but does reduce airway eosinophilia and normalizes the hypertussive state. The effects of Compound I on direct antitussive activity are shown in FIG. 4. Similarly, inhalation of LTD4 (10 ug/ml for 1 minute) increased cough incidence and airway esoinophiles 72 hours after exposure. Oral treatment with Compound 1 (30 mg/kg, b.i.d.), blocked the LTD4-induced esoinophilia, and normalized the cough response, as shown in FIG. 5 herein.

These findings provide evidence of the role of eosinophils in the maintenance of increased cough responsiveness and support the utility of and efficacy of using a p38 MAP kinase inhibitors for this type of therapy.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claimed is:

1. A method of treatment, including prophylaxis, for inflammation enhanced cough in a mammal in need thereof, which comprises administering to said mammal an effective amount of a CBSP/p38 inhibitor.

2. The method according to claim 1 wherein the inflammation enhanced cough is cough variant asthma.

3. The method according to claim 1 wherein the inflammation enhanced cough is eosinophilic bronchitis.

4. The method according to any one of claims 1 to 3 wherein the CSBP/p38 inhibitor is administered with a second therapeutic agent.

5. The method according to claim 4 wherein the second therapeutic agent is an anti-tussive; an antihistamine; a steroid; a PDE₄ agent, an antibiotic; or an antiinflammatory agent selected from an NSAID, a COX-1 or COX-2 inhibitor, ASA, or indomethacin.

6. The method according to any one of claims 1 to 3 wherein the therapeutic agent is administered orally, topically (intranasal) or via inhalation (aerosol), or both topically and via inhalation.

7. The method according to claim 6 wherein the CSBP/p38 inhibitor is administered with a second therapeutic agent.

8. The method according to claim 7 wherein the second therapeutic agent may be administered by a different route than the CSBP/p38 inhibitor.

9. The method according to any one of claims 1 to 3 wherein the CSBP/p38 inhibitor is selected from a compound disclosed in U.S. Pat. Nos. 5,716,972, 5,686,455, 5,656,644, 5,593,992, 5,593,991, 5,663,334, 5,670,527, 5,559,137, 5,658,903, 5,739,143, 5,756,499, 5,716,955, WO 98/25619, WO 97/25048, WO 99/01452, WO 97/25047, WO 99/01131, WO 99/01130, WO 97/33883, WO 97/35856, WO 97/35855, WO 98/06715, WO 98/07425, WO 98/28292, WO 98/56377, WO 98/07966, WO 99/01136, WO 99/17776, WO 99/01131, WO 99/01130, WO 99/32121, WO 00/26209, WO 99/58502, WO 99/58523, WO 99/57101, WO 99/61426, WO 99/59960, WO 99/59959, WO 00/18738, WO 00/17175, WO 99/17204, WO 00/20402, WO 99/64400, WO 00/01688, WO 00/07980, WO 00/07991, WO 00/06563, WO 00/12074, WO 00/12497, WO 00/31072, WO 00/31063, WO 00/23072, WO 00/31065, WO 00/35911, WO 00/39116, WO 00/43384, WO 00/41698, WO 97/36587, WO 97/47618, WO 97/16442, WO 97/16441, WO 97/12876, WO 98/7966, WO 98/56377, WO 98/22109, WO 98/24782, WO 98/24780, WO 98/22457, WO 98/52558, WO 98/52941, WO 98/52937, WO 98/52940, WO 98/56788, WO 98/27098, WO 99/00357, WO 98/47892, WO 98/47899, WO 99/03837, WO 99/01441, WO 99/01449, WO 99/03484, WO 95/09853, WO 95/09851, WO 95/09847, WO 95/09852, WO 92/12154, WO 94/19350, WO 99/15164, WO 98/50356, DE 19842833, or JP 2000 86657.

10. The method according to any one of claims 1 to 3, or 9 wherein the compound is 1-(1,3-Dihydroxyprop-2-yl)-4-(4-fluorophenyl)-5-(2-phenoxypyrimidin-4-yl)imidazole, or a pharmaceutically acceptable salt thereof.

11. The method according to any one of claims 1 to 3, or 9 wherein the compound is trans-1-(4-Hydroxycyclohexyl)-4-(4-fluorophenyl)-5-[(2-methoxy)pyrimidin-4-yl]imidazole; 1-(4-Piperidinyl)-4-(4-fluorophenyl)-5-(2-methoxy-4-pyrimidinyl)imidazole; or (4-Fluorophenyl)-2-(4-methylsulfinylphenyl)-5-(4-pyridyl)-imidazole.

12. The method according to claim 1 or 9 wherein the compound is VX-745, RWJ 67657, RWJ-68354, ZM 336372, SU 4984 or RPR-200765A.
